(19) United States
(12) Patent Application Publication
(10) Pub. No.: US 2007/0054322 A1
(43) Pub. Date: Mar. 8, 2007

(54) Methods and kits for the detection of prion diseases

(75) Inventor: Ruth Gabizon, Jerusalem (IL)

Correspondence Address: BANNER & WITCOFF, 1001 G STREET N W, SUITE 1100, WASHINGTON, DC, 20001, US

(73) Assignee: HADASIT MEDICAL RESEARCH SERVICES & DEVELOPMENT LT, JERUSALEM (IL)

(21) Appl. No.: 10/566,279

(22) PCT Filed: Jul. 29, 2004
(86) PCT No.: PCT/IL04/00699

§ 371 Date: Oct. 10, 2006

Related U.S. Application Data
(60) Provisional application No. 60/491,237, filed on Jul. 31, 2003.

(51) Int. Cl. G01N 33/567

(52) U.S. Cl. 435/7.2

(57) ABSTRACT

The present invention relates to a method for the diagnosis of a neurodegenerative disorder in a mammalian subject. The method of the invention comprises the steps of (a) providing a body fluid sample of said subject; (b) concentrating proteins comprised within said sample by a suitable means; (c) contacting the concentrated sample obtained in step (b) with a sufficient amount of a protein which has a beta-sheet structure, preferably, an IgG light chain, under conditions suitable to allow the formation of aggregates. Which aggregates comprise a protein associated with a neurodegenerative disorder, and (d) measuring aggregate formation by suitable means. Whereby the presence of aggregates in said sample indicates that said subject carries said neurodegenerative disorder. The invention further provides for kits and diagnostic compositions for the detection of a neurodegenerative disorder, particularly, prion diseases.

### FIELD OF THE INVENTION

The present invention relates to methods for the diagnosis of a neurodegenerative disorder in a mammalian subject. More particularly, the invention relates to methods and kits for the diagnosis of prion diseases by detection of PrP^{SC} aggregates in urine samples. The diagnostic methods of the invention are based on enhancing the formation of PrP^{SC} aggregates by the addition of a protein which has a beta-sheet structure, to a test sample, and detecting the formation of aggregates using a suitable means.

### BACKGROUND OF THE INVENTION

Throughout this application various publications are referenced to. It should be appreciated that the disclosure of these publications, including references cited therein in their entireties, is hereby incorporated into this application in order to more fully describe the state of the art as known to those skilled therein as of the date of the invention described and claimed herein.

Prion diseases, also known as TSEs (transmissible spongiform encephalopathies), are a group of fatal neurodegenerative diseases of animals and humans. Among the animal diseases, the most prevalent today is BSE (bovine spongiform encephalopathy) also known as the “Mad Cow Disease”. Although less than 100 patients have been diagnosed to date to be BSE-infected, the number of individuals incubating the disease may be millions. Another animal prion disease is scrapie in sheep, which after transmission to rodents constitutes the main experimental prion animal model.

In humans, the most prevalent prion disease is CJD (Creutzfeldt Jakob Disease), which can be manifested either sporadically (about 1 patient per year); genetically (via mutations in the prion protein PrP gene); or in transmissible form, as in the BSE affected cases. It is a well known experimental fact that the incubation of prion diseases in humans and large animals can last decades.

Prion diseases are believed to be caused by the accumulation in the brain of PrP^{SC}, an abnormally folded isoform of PrP^{C}, a GPI anchored protein of unknown function. It has been postulated that prion diseases propagate by the conversion of PrP^{C} molecules into protease-resistant and insoluble PrP^{SC} by an as yet unknown mechanism. The proteinase K (PK) resistant PrP in prion diseases was described by McKinley et al. [Cell 35(1):57-62 (1983)]. Immunoblotting of a Proteinase K-digested brain sample infected with a prion disease with an anti-PrP antibody, reveals a characteristic N-terminally truncated PrP protein (the protease resistant core of PrP^{SC}, denominated PrP 27-30), which is not present in controls or in individuals affected with any other neurological disease.

Diagnosis of prion diseases was based on the presence of this characteristic protease-resistant PrP in brain biopsies, as well as on clinical criteria. Current methods for the conclusive identification of Prion diseases include mostly a post-mortem analysis of the subject's brain homogenate. Clinical symptoms of the disease can many times be misleading. Evidently, sampling brain tissue from the living subject or patient involves a painful and risky surgical procedure and, moreover, does not give a definite answer since the distribution of PrP^{SC} in the brain is not homogenous. All commercial tests used to date are based on brain presence of protease resistant PrP, for example the Prion-Test of Prionics AG, Switzerland (which company is in charge of most European active surveillance for BSE cases), which is an immunological test for the detection of prions in brain and spinal cord tissue, and is mainly used for BSE and scrapie diagnostics. Since the incubation period in prion diseases is very long (years), it is possible that there is a large number of asymptomatic human and animal carriers. There exists therefore a need for developing a simple and readily available pre-clinical and clinical diagnostic non-invasive test for the disease. The need for such an in-vivo test has been reinforced since the reports of the first cases of variant Creutzfeldt Jakob disease (vCJD) in 1996 [Zeidler, M. et al., Lancet 350(9082):908-10 (1997); Bruce, M. E. et al., Nature 389(6650):498-501 (1997); Ironside, J. W. et al., Histopathology 37(1):1-9 (2000)]. vCJD is a fatal neurodegenerative disease believed to be caused by the consumption of BSE contaminated meat, and the incubation time between infection to clinical symptoms may be as long as decades [Bruce (1997) ibid.]. As opposed to cattle, the incubating individuals will be present for many years, donating blood and in some cases other organs to the non-affected population. Additionally, such test is important for the food industry, and would enable detecting BSE in bovine animals such as cows and sheep, and to prevent marketing of infected meat and dairy products of these animals.

Therefore, a major object of the present invention is the development of a reliable, non-invasive method for diagnosing prion diseases which will allow the pre-clinical and clinical diagnosis of the disease in humans and in animals.

Since most urine proteins originate from the blood, the present inventors speculated that some PrP^{SC}, either from brain or from a peripheral organ, is released during the incubation period into the blood serum in a non-aggregated form, although at low and undetectable concentrations. Due to its protease resistance, PrP^{SC} is not digested by blood proteases. However, since the MW of PrP is below the cutoff size for filtering through kidney cells (about 40 kDA) [Berne, R. M. and Levy, M. N. Physiology 4th Ed (1998)], PrP may subsequently be secreted into the urine and thereby be concentrated, as other proteins, at about 120 folds of its concentration in blood [Kocisko, D. A. et al., Nature 370(6489):471-4 (1994)]. The concentration by the kidney makes possible to detect PrP^{SC} in urine more easily than in blood.

The present inventors have previously developed a method for the detection of the protease resistance abnormal isoform of the prion protein, PrP^{SC}, in a urine sample [WO 02/33420]. The procedure described in this publication is based on the enrichment of the protease resistant isoform in the urine sample by dialyzing the sample through membrane having a pore range of about 6 Kd-8 Kd, followed by protease digestion and immunological assay.

The theoretical possibility for diagnosis of prion diseases in a variety of body fluids, such as urine, has been mentioned in several patent documents. EP 0854364, for example, discloses a diagnostic method for neuro-degenerative disorders such as Alzheimer's disease and prion diseases. This method is based on concentrating a protein associated with the specific neuro-degenerative disease (such as PrP in prion diseases and APP in Alzheimer's disease) in a sample (urine, for example). The concentration is carried out by contacting the sample with a solid, non-buoyant particulate material having free ionic valencies such as calcium phosphate. However, this patent exemplifies the detection of only the Alzheimer's disease associated peptide APP. WO 93/23432 discloses a diagnostic method for prion diseases in different body fluids such as CSF (cerebrospinal fluid) and theoretically, urine. Similarly to EP 0854364, this method is based on concentrating the prion protein by ammonium sulfate precipitation and affinity chromatography. This publication exemplifies only CSF as a sample.

Recently, Soto et al., [Saborio, et al., Nature 441:810-813 (2001)] have reported the development of a method for the detection of prion protein by cyclic amplification of protein misfolding (PMCA). This method is based on the rapid conversion of large excess PrP^{C} into the protease-resistant PrP^{SC} like form in the presence of minute quantities of PrP^{SC} template which exist in a positive sample. In this procedure, aggregates formed when PrP^{SC} is incubated with PrP^{C}, are disrupted by sonication to generate multiple smaller units for the continued formation of new PrP^{SC}. According to this method, a tested sample diluted brain homogenate of a scrapie-affected hamster was incubated with brain homogenate from healthy hamsters as a source of PrP^{C}. Following cycles of incubation-sonication, the prion protein was then detected using an immunoassay (blot incubated with a PrP^{SC} specific antibody).

As will become apparent as the description proceeds, the present inventors have developed a simple, non-invasive, rapid and protease free method for the detection of PrP^{SC} aggregates in urine samples of prion infected animals and humans (UPrP^{SC}). More particularly, the diagnostic method of the invention enhances the aggregation of the abnormal isoform of PrP in an infected sample, and therefore enables the detection of these aggregates in urine samples obtained from cattle and sheep, without using prion specific antibodies. The diagnostic method of the invention may be used for the in-vivo early diagnosis of ill as well as seemingly healthy but prion infected individuals. Moreover, the present invention shows that the abnormal prion protein isoform PrP^{SC} may be detected, following a specific aggregate enhancement procedure, in the urine of scrapie-infected sheep, BSE-infected cattle and humans suffering from CJD. This specific enhancement procedure according to the present invention includes the addition of a protein which has a beta-sheet structure to the tested sample, preferably IgG light chain (LC), Bence Jones protein (BJ) or recombinant PrP, most preferably, IgG light chain (LC), prior to a staining procedure using Congo Red. The sensitive and specific method shown by the invention, strongly suggests that it may be used also for pre-clinical diagnosis.

Thus, the specific enhancement of formation of aggregates comprising a prion protein in a urine sample, according to the present invention, provides a novel, sensitive and reliable method for the detection of different prion diseases by a non-invasive procedure.

### SUMMARY OF THE INVENTION

In a first aspect, the present invention relates to a method for the diagnosis of a neurodegenerative disorder in a mammalian subject comprising: (a) providing a body fluid sample of said subject; (b) concentrating proteins comprised within the sample, by a suitable means; (c) contacting the concentrated sample obtained in step (b) with a sufficient amount of a protein which has a beta-sheet structure, under conditions suitable to allow the formation of aggregates, which aggregates comprise a protein associated with a neurodegenerative disorder; and (d) measuring aggregate formation by suitable means, whereby the presence of aggregates in the sample indicates that the tested subject carries a neurodegenerative disorder.

According to one embodiment, the measurement of aggregate formation in said step (d), may comprise the following steps: (i) adding to the mixture obtained in step (c) a binding material capable of binding aggregates of proteins associated with said neurodegenerative disorder; (ii) applying the sample obtained in step (i) onto a solid support; and (iii) detecting a visual signal indicating the presence of aggregates comprising a neurodegenerative disorder associated protein in the tested sample.

Optionally, the method of the invention may comprise a further step of separating said aggregates from the mixture obtained in step (c) by suitable means, prior to the addition of the binding material. According to a specific embodiment, such separation may be performed by any one of proteinase K digestion, dialysis or centrifugation.

In another preferred embodiment, the binding material may be selected from the group consisting of an antibody, a peptide, a substance having affinity to a specific compound in said aggregate and a specific dye. Preferably, such specific dye may be any one of Congo Red, Thioflavin-T and Thioflavin-S. Most preferably, the binding material used is Congo Red.

In another alternative embodiment, specifically were the method comprises the further step of aggregate separation, the binding material may be an antibody which specifically recognizes the protein which has a beta-sheet structure.

In another specific embodiment, the protein which has a beta-sheet structure used by the method of the invention may be selected from the group consisting of IgG light chain (LC), human Bence Jones (BJ) protein and recombinant PrP protein. Most preferred beta-sheet protein may be IgG light chain (LC).

The method of the invention is intended for diagnosis of a neurodegenerative disorder such as Alzheimer's disease, multiple sclerosis, or spongiform encephalopathy. More specifically, spongiform encephalopathy may be any one of Creutzfeldt-Jakob disease (CJD), Gerstmann-Straussler-Scheinker Syndrome (GSS), Kuru, scrapie and bovine spongiform encephalopathy (BSE).

Still further, the method of the invention is particularly applicable for mammalian subjects such as humans, sheep, goats, bovines, minks, hamsters and felines such as cats.

The body fluid sample used by the method of the invention may be a sample of blood, lymph, milk, urine, faeces, semen, brain extracts, spinal cord fluid (SCF), appendix, spleen and tonsillar tissue extracts samples. Preferred sample may be a urine sample.

It should be noted that according to a specific embodiment, the proteins in the tested sample are concentrated by centrifugation and precipitation.

According to another specific embodiment, where the neurodegenerative disorder diagnosed by the method of the invention is a prion disease, the neurodegenerative disease associated protein may be the abnormal isoform of prion protein (PrP^{SC}).

The present invention thus provides a method for the diagnosis of a spongiform encephalopathy, which is a prion disease in a mammalian subject. According to a particular embodiment, this method comprises: (a) providing a urine sample of said subject; (b) concentrating proteins comprised within the sample by centrifugation and precipitation or preferably by using Amicon tubes; (c) contacting the concentrated sample obtained in step (b) with a sufficient amount of IgG LC, under conditions suitable to allow the formation of aggregates, which aggregates comprise the abnormal isoform of prion protein (PrP^{SC}); (d) adding Congo Red to the sample mixture obtained in step (c), in an amount sufficient for detection of aggregates comprising the abnormal isoform of the prion protein (PrP^{SC}); (e) applying the sample obtained in step (d) onto a nitrocellulose membrane; and (f) detecting a visual signal indicating the presence of aggregates comprising the abnormal isoform of prion protein (PrP^{SC}) in the tested urine sample; whereby the presence of aggregates in the sample indicates that the tested subject may carry a prion disease.

It should be appreciated that diagnosis of prion disease according to the method of the invention may be performed prior to or after onset of clinical symptoms.

In a further aspect, the present invention relates to a method for detecting the presence of a neurodegenerative disorder associated protein in a sample of a subject, which method comprises the steps of: (a) providing a body fluid sample of the tested subject; (b) concentrating proteins comprised within the sample by a suitable means, preferably by using Amicon tubes or alternatively, by centrifugation and precipitation; (c) contacting the concentrated sample obtained in step (b) with a sufficient amount of a protein which has a beta-sheet structure, under conditions suitable to allow the formation of aggregates comprising the neurodegenerative disorder associated protein; and (d) measuring aggregate formation by suitable means.

According to a particular and specific embodiment, the invention provides for a method for detecting the presence of the abnormal isoform of prion protein (PrP^{SC}) in a urine sample of a subject. This method comprises the steps of: (a) providing a urine sample of the tested subject; (b) concentrating proteins comprised within the sample preferably by using Amicon tubes or alternatively, by centrifugation and precipitation; (c) contacting the concentrated sample obtained in step (b) with a sufficient amount of IgG LC, under conditions suitable to allow the formation of aggregates, which aggregates comprise the abnormal isoform of prion protein (PrP^{SC}); (d) adding Congo Red to the sample mixture obtained in step (c), in an amount sufficient for detection of formation of aggregates comprising the abnormal isoform of prion protein (PrP^{SC}); (e) applying the sample obtained in step (d) onto a nitrocellulose membrane; and (f) detecting a visual signal indicating the presence of aggregates comprising the abnormal isoform of prion protein (PrP^{SC}) in the tested urine sample; whereby the presence of aggregates in the analyzed sample is indicative of the presence of the abnormal isoform of prion protein (PrP^{SC}) in the analyzed sample.

In a third aspect, the present invention relates to kit for the diagnosis of a neurodegenerative disorder in a mammalian subject, such kit may comprise: (a) means for obtaining a sample from a tested mammalian subject; (b) means for concentrating proteins in the tested sample; (c) a protein which has a beta sheet structure; (d) means for measuring aggregate formation in the sample; (e) optionally, suitable buffers; and (f) instructions for carrying out the detection of the presence of aggregates comprising a neurodegenerative disorder associated protein in the tested sample.

According to one preferred embodiment, the kit of the invention may optionally further comprise means for separating the aggregates from the sample prior to measuring aggregate formation.

According to another preferred embodiment, the kit of the invention may be designed for the diagnosis of a neurodegenerative disorder in a mammalian subject according to the methods of the invention.

Specifically preferred kit according to the invention is particularly applicable for the detection of a prion disease in a mammalian subject, using a urine sample. Such specific kit may preferably comprise means for obtaining a urine sample from the tested subject, IgG light chain (LC), as a protein which has a beta-sheet structure, Congo Red, solid support for attachment of proteins in said sample (for example nitrocellulose membrane), further optional buffers and instructions for carrying out the detection of the presence of aggregates comprising the abnormal prion protein in the tested urine sample.

The invention further provides for the use of a protein which has a beta-sheet structure, preferably, IgG light chain, which enhances the formation of aggregates comprising a neurodegenerative disease associated protein, in the preparation of a diagnostic composition for the diagnosis of a neurodegenerative disorder.

According to another preferred embodiment, where the neurodegenerative disease to be diagnosed is spongiform encephalopathy, the disease associated protein may be the abnormal isoform of prion protein (PrP^{SC}).

Therefore, the invention provides for a diagnostic composition for the detection of a neurodegenerative disease in a mammalian subject, preferably, spongiform encephalopathy. Such composition comprises as an effective ingredient a sufficient amount of a protein which has a beta-sheet structure, preferably, IgG light chain.

The invention will be further described on the hand of the following figures, which are illustrative only and do not limit the scope of the invention which is defined by the appended claims.

### BRIEF DESCRIPTION OF THE INVENTION

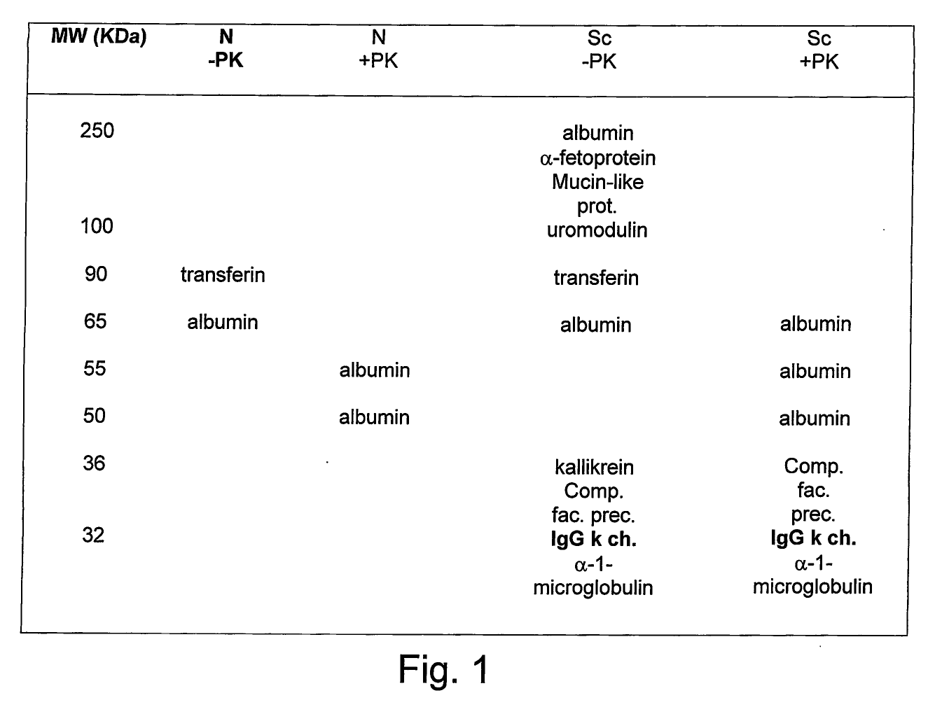

FIG. 1 Prion Urine Contains Protease Resistant IgG Light Chain (LC)

Concentrated urine samples obtained from normal and scrapie infected urine, before and after PK digestion, were separated on 12% SDS PAGE and stained with Coomassie Blue. Bands were cut from the gel and sent to sequencing by mass spectrometry. The resulting protein profile of each sample is illustrated in the table. Abbreviations: N (normal urine sample), Sc. (scrapie urine sample), MW (molecular weight), PK (proteinase K), comp. fac. Prec. (complement factor precursor), Ch. (chain).

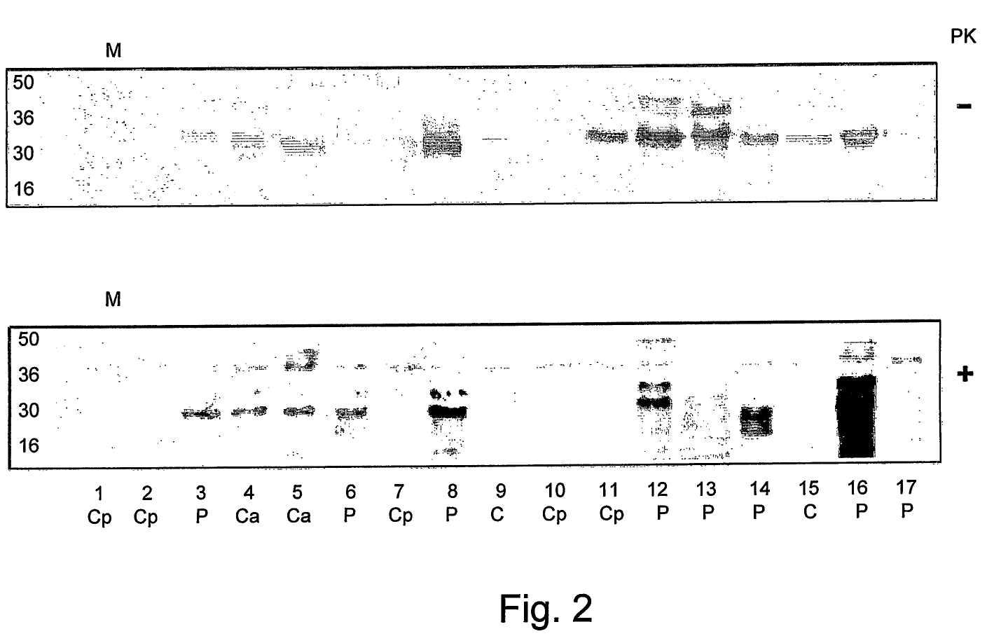

FIG. 2 CJD Urine can be Identified Using Anti Human IgG Antibody

Urine from patients and controls were concentrated by minicon and digested in the presence or absence of PK. Samples were subjected to 12% SDS-PAGE and immunoblotted using AP conjugated anti-human IgG antibody. Abbreviations: P (patient), CP (non CJD neurological patient), C (normal control), Ca (healthy carrier of the E200K mutation), PK (proteinase K), M. (marker).

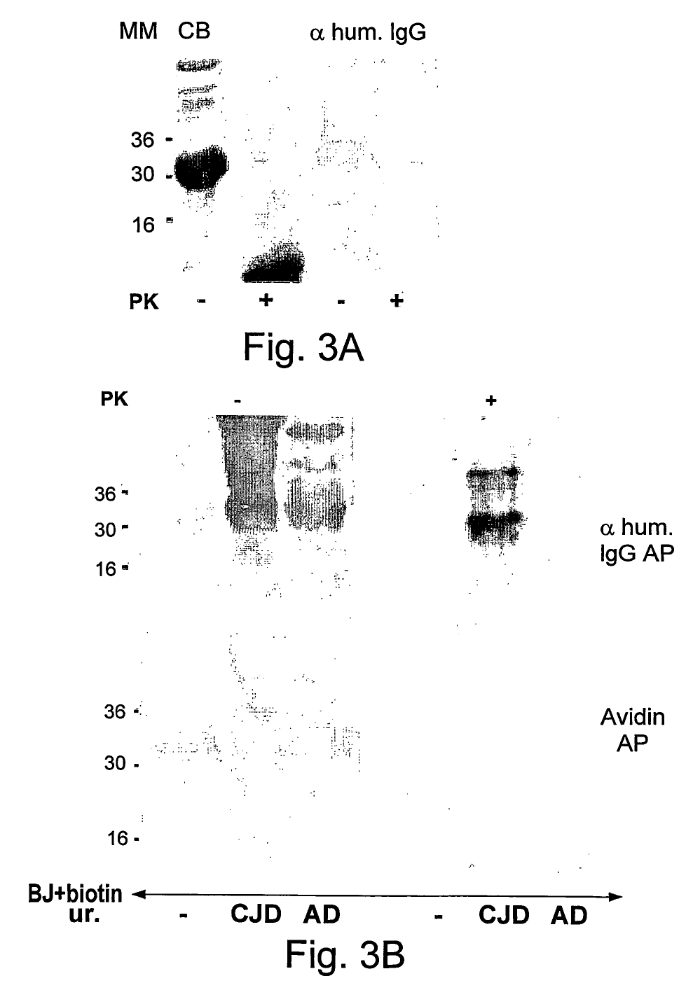

FIGS. 3A-3B Bence Jones (BJ) Protein is Converted into Protease Resistant Protein by CJD Urine

FIG. 3A shows Coomassie Blue (CB) staining and Western blot of urine obtained from Multiple Myeloma patient. The samples were concentrated by minicon and digested in the presence or absence of 20 μg/ml PK for 30 min. at 37° C. Samples were subjected to 12% SDS PAGE and either stained with CB or immunoblotted with anti human IgG antibody.

FIG. 3B shows Western blot analysis of biotinylated purified BJ protein samples that were incubated overnight alone, or in the presence of 5 ml CJD or AD urine. Subsequently, samples were concentrated by minicon and digested in the presence or absence of PK. Blots were immunoblotted either with anti human IgG (upper panels) or with Avidin AP Gower panels). Abbreviations: CB (Coomassie Blue), α-hum. IgG (anti human immunoglobulin G antibody), PK (proteinase K), MW (molecular weight), AD (urine samples of Alzheimer disease patient), CJD (urine sample of Creutzfeldt Jakob disease patient), Ur. (urine).

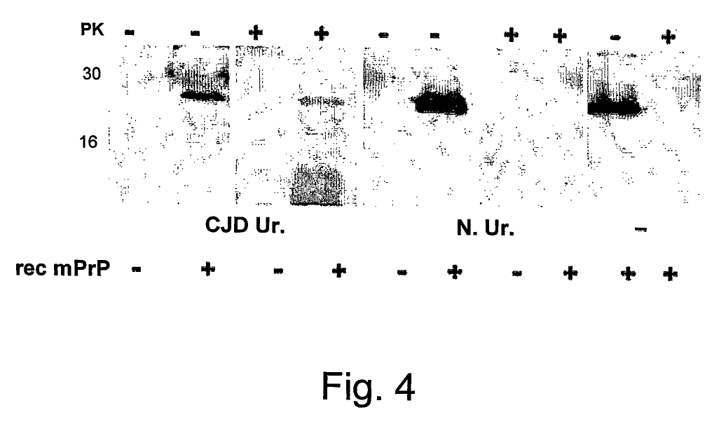

FIG. 4 Recombinant PrP Becomes Protease Resistant in the Presence of CJD Urine

Western blot analysis of mouse recombinant PrP samples which were incubated overnight with concentrated normal or CJD urine, digested in the presence of PK and subsequently separated on 14% SDS PAGE. The blot was incubated with anti PrP mAb 6H4. Abbreviations: PK proteinase K), CJD (urine sample of Creutzfeldt Jakob disease patient), Ur. (urine), N (normal) rec mPrP (recombinant PrP).

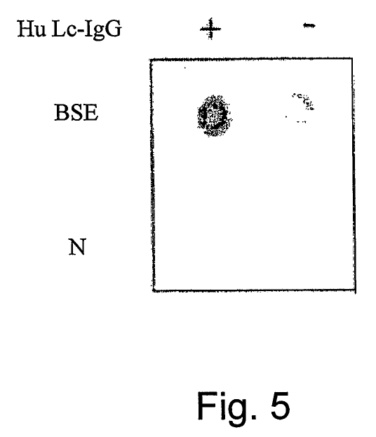

FIG. 5 LC Enhances Aggregate Formation in BSE Sample

Congo Red staining of dot blot analysis of samples obtained from prion infected BSE or normal bovine, with or without human IgG LC (human immunoglobulin G light chain).

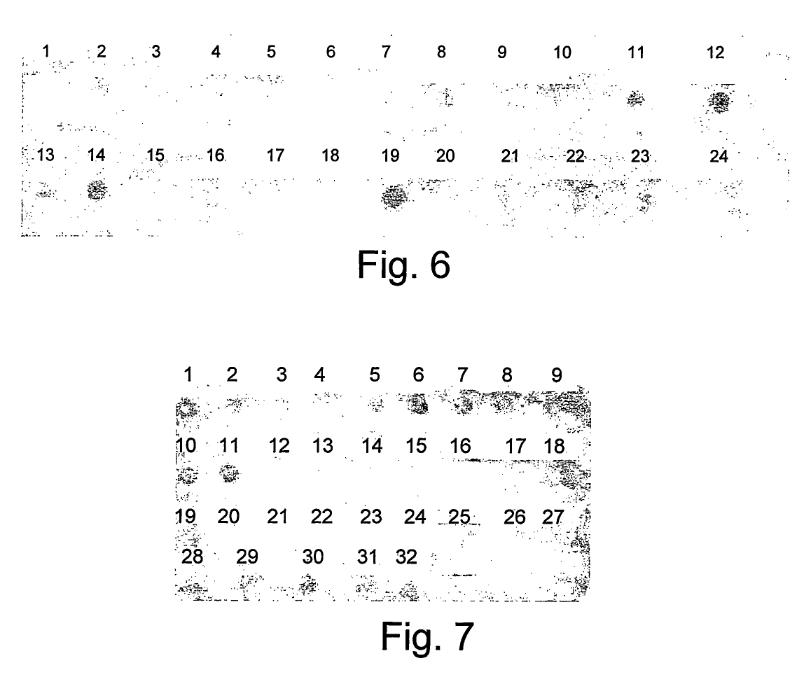

FIG. 6 LC Enhances Aggregate Formation in CJD Samples

Congo Red staining of dot blot analysis of samples obtained from CJD patients and family members of one of the patients. Samples 1, 12, 19, and 23 were obtained from patients, samples 11, 21 and 22 from suspected patients, samples 2, 7, 8, 13, 14, and 16 from mutation carriers, samples 3, 4, 5, 6, 10, 15, 18 and 20, from family members non carriers subjects and samples 24 and 17 were obtained from healthy controls.

FIG. 7 LC Enhances Aggregate Formation in Scrapie Sample

Congo Red staining of dot blot analysis of normal and scrapie infected sheep. Samples 1, 2, 5, 6, 7, 8, 9, 10, and 11 were obtained from reported positive sheep, samples 21-24, 3 and 4 negative from clean herd, samples 13-20 were obtained from negative sheep from infected flock and samples 25-32 were obtained from suspected samples.

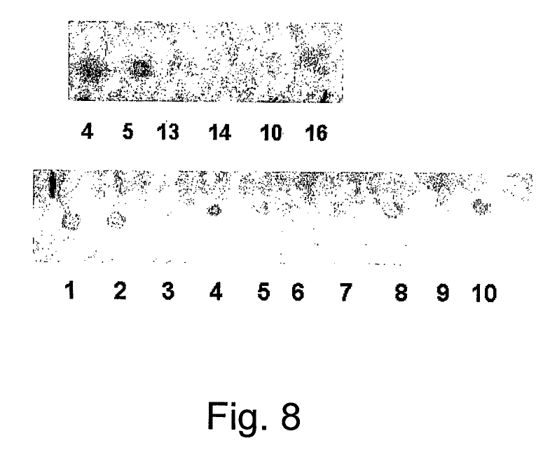

FIG. 8 Congo Red Staining of Normal and BSE Infected Cows

Congo Red staining of dot blot analysis of urine samples obtained from normal and infected cows. Samples 2, 4, 5, 8, 10 and 16 were obtained from reported positive cows.

### DETAILED DESCRIPTION OF THE INVENTION

Extensive evidence has accumulated indicating that several diverse disorders have the same molecular basis, i.e. a change in a protein conformation [Thomas et al., Trends Biochem. Sci. 20: 456-459, (1995); Soto, J. Mol. Med. 77:412-418 (1999)]. These protein conformational diseases include Alzheimer's disease, systemic amyloidosis, Huntington's disease, prion-related disorders (also known as transmissible spongiform encephalopathy), and Amyotrophic Lateral Sclerosis [Soto (1999) ibid.]. The hallmark event in protein conformational disorders is a change in the secondary and tertiary structure of a normal protein without alteration of the primary structure. The conformationally modified protein may be implicated in the disease by direct toxic activity, by the lack of biological function of normally-folded protein, or by improper trafficking [Thomas (1995) ibid.]. In cases where the protein is toxic, it usually self-associates and becomes deposited as amyloid fibrils in diverse organs, inducing tissue damage [Thomas (1995) ibid.; Kelly, Curr. Opin. Struct. Biol. 6:11-17 (1996); Soto (1999) ibid.].

Amyloid is a generic term that describes fibrillar aggregates that have a common structural motif, i.e., the β-pleated sheet conformation [Serpell, et al., Cell Mol. Life Sci. 53:887 (1997); Sipe, et al., Ann. Rev. Biochem. 61:947-975 (1992)]. These aggregates exhibit specific properties, including the ability to emit a green glow after staining with Congo Red, and the capacity to bind the fluorochrome, thioflavin S [Sipe (1992) ibid.; Ghiso, et al., Mol. Neurobiol. 8:49-64 (1994)].

The formation of amyloid is basically a problem of protein folding, whereby a mainly random coil soluble peptide becomes aggregated, adopting a beta-pleated sheet conformation Kelly (1996) ibid.; Soto (1999) ibid.]. Amyloid formation proceeds by hydrophobic interactions among conformationally altered amyloidic intermediates, which become structurally organized into a beta-sheet conformation upon peptide interaction. The hydrophobicity appears to be important to induce interaction of the monomers leading to aggregation, while the beta-sheet conformation might determine the ordering of the aggregates in amyloid fibrils.

Spongiform encephalopathy diseases, which are also known as prion diseases are associated with the accumulation of a conformational isomer (PrP^{SC}) of host-derived prion protein (PrP) with an increase in its beta-sheet content. According to the protein-only hypothesis, PrP^{SC} is the principal or sole component of transmissible prions. Although the structure of PrP^{C} has been determined and has been found to consist predominantly of α-helices, the insolubility of PrP^{SC}, which is isolated from tissue in a highly aggregated state and which has a high beta-sheet content, has precluded high-resolution structural analysis. Various publications [e.g. Hornernann and Glockshuber Proc. Natal. Acad. Sci. USA 95:6010-6014 (1998)] describe a β-intermediate which is an unfolding intermediate of mouse PrP and contains predominantly β-sheet elements of secondary structure as opposed to α-helix. Chemical differences have not been detected to distinguish between PrP isoforms and the conversion seems to involve a conformational change whereby the α-helical content of the normal protein diminishes and the amount, of beta-sheet increases. The structural changes are followed by alterations in the biochemical properties: PrP^{C} is soluble in non-denaturing detergents, PrP^{SC} is insoluble; PrP^{C} is readily digested by proteases, while PrP^{SC} is partially resistant, resulting in the formation of a N-teminally truncated fragment.

Prion diseases are characterized by an extremely long incubation period, followed by a brief and invariably fatal clinical disease. To date no therapy is available.

As mentioned above, the present inventors have previously established a method for the detection of the abnormal prion protein in urine samples, which was based on specific enrichment procedure including dialysis of the urine sample through membrane having pore range of about 6 KD to about 8 Kd, followed by protease digestion and immunoassay [WO 02/33420]. The inventors have now surprisingly found, and this is an object of the invention, that PrP^{SC}, the aberrant isoform and the only known marker for prion diseases, can be identified in the urine of sheep infected with scrapie, cows infected with BSE, as well as in the urine of humans sick with CJD, using a rapid, sensitive and specific methods, preferably, methods avoiding dialysis, ultracentrifugation, protease digestion and immunological detection steps. The method of the invention is based on enhancement of aggregate formation in a sample taken from prion infected subject, by addition of IgG light chain. Formation of aggregate may then be measured by any known method, such as Congo Red staining followed by dot blot analysis.

The invention thus provides an efficient, non-invasive method for the diagnosis of prion diseases. It may be appreciated that while the rationale underlying the method of the present invention is yet unclear, it is possible that the PrP^{SC} is secreted from the brain cells during the pre-clinical or clinical stage of the disease, and since this protein is protease-resistant, it is cleared into the urine before it can be digested in the blood.

Thus, in a first aspect, the present invention relates to a method for the diagnosis of a neurodegenerative disorder in a mammalian subject comprising: (a) providing a body fluid sample of said subject; (b) concentrating proteins comprised within the sample, by a suitable means; (c) contacting the concentrated sample obtained in step (b) with a sufficient amount of a protein which has a beta-sheet structure. Such contact should be performed under conditions suitable to allow the formation of aggregates. Such aggregates comprise a protein associated with a neurodegenerative disorder; and (d) measuring aggregate formation by suitable means, whereby the presence of aggregates in the sample indicates that the tested subject carries said neurodegenerative disorder.

In another embodiment, the present invention relates to a method for the diagnosis of a neurodegenerative disorder in a mammalian subject comprising: (a) concentrating proteins comprised within a body fluid sample of said subject, by a suitable means; (b) contacting the concentrated sample obtained in step (a) with a sufficient amount of a protein which has a beta-sheet structure. Such contact should be performed under conditions suitable to allow the formation of aggregates. Such aggregates comprise a protein associated with a neurodegenerative disorder; and (c) measuring aggregate formation by suitable means, whereby the presence of aggregates in the sample indicates that the tested subject carries said neurodegenerative disorder.

Preferably, the aggregates which are formed in accordance with the invention may be non-covalent or non-cross linked aggregates. Covalent aggregates may be formed by disulphide bridges between two cysteine residues present in different proteins. Non-covalent aggregates are not necessarily formed through disulphide bonds, but e.g. through adoption of a high β-structure content.

Aggregation typically occurs by nucleation. Nucleation occurs when intermolecular bonds form between polypeptides in a partially or fully denatured state. The process of nucleation can therefore be brought about in any situation by contacting polypeptides, preferably, a protein which has a beta-sheet structure, under suitable conditions. For the purposes of nucleation, a suitable condition is one under which partially or fully denatured polypeptide molecules are generated, although conditions must not favor denaturation to the extent that intermolecular bonds are prevented from forming. The optimal nucleation conditions are different for each polypeptide. Important parameters for nucleation typically include variations in solvents, polypeptide concentration, salt, ligands, temperature and pH. A skilled person will be able to determine suitable conditions for any given protein which has a beta-sheet structure. According to the invention and as indicated in the following Examples, nucleation can be caused by incubation of the sample with a protein which has a beta-sheet structure in STE+Sarcosyl, preferably at 10% to 0.5% concentration, for example 5% to 1%, preferably around 2%, preferably for at least 1 hour in room temperature, or overnight in 4° C.

In some cases, it may be advantageous to alter the optimal nucleation conditions for any given protein which has a beta-sheet structure.

Aggregate formation, such as fibril formation, can be measured by any technique known in the art. Techniques typically used include circular dichroism, sedimentation analysis, Thioflavin-T and Congo Red binding assays, polypeptidase resistance assays, Fourier-transform infrared spectroscopy, electron microscopy and X-ray diffraction.

It should be noted that aggregation assays do not require the step of separation of the two isoforms, it because is known that normal PrP^{C} does not aggregate.

Therefore, according to one embodiment, measurement of aggregate formation in said step (d), may comprise the following steps: (i) adding to the mixture obtained in step (c), a binding material capable of binding aggregates of proteins associated with a neurodegenerative disorder; (ii) applying the sample obtained in step (i) onto a solid support; and (iii) detecting a visual signal indicating the presence of aggregates comprising a neurodegenerative disorder associated protein in the tested sample.

As used herein, a “binding material capable of binding aggregates of protein associated with neurodegenerative disorders” includes any material such as a protein, a peptide, sequence of either, an antibody, a substance having affinity to a specific compound in said aggregate, a specific dye such as Congo Red, Thioflavin-T, or any species capable of the binding so described. In the case of antibodies, this binding is site-specific. In other cases, it can be non-specific. In the case of proteins or peptides, the binding typically involves non-specific β-sheet/β-sheet interactions. Binding species can also include peptides, fragments, or whole proteins that are homologous to naturally-occurring neurodegenerative disease aggregate.

In another preferred embodiment, the binding material may be a specific dye such as Congo Red, Thioflavin-T or Thioflavin-S. Preferably, the binding material used is Congo Red.

“Proteins associated with neurodegenerative disease comprised within aggregates”, as used herein, means proteins associated with neurodegenerative disease having sufficient binding capacity to bind to other molecules associated with neurodegenerative disorder (including like molecules), to form fibrils or aggregates characteristic of neurodegenerative disease. Such aggregate-forming proteins typically are characterized by a change in molecule conformation, relative to sequence-homologous, healthy counterparts, allowing them to bind more readily to like or similar molecules. In some cases, such aggregate-forming proteins have the capability to convert proteins from non-aggregate-forming conformation into aggregate-forming conformation.

In another specific embodiment, the protein which has a beta-sheet structure used by the method of the invention for enhancing aggregate formation may be any one of IgG light chain (LC), recombinant human Bence Jones (BJ) protein and recombinant PrP protein, preferably, IgG light chain (LC).

It should be noted that any other proteins which has a beta-sheet structure may be applicable for the method of the invention, for example, a T cell receptor or any fragments thereof, β2-microglobulin, transthyrein (a tetramer where each subunit is composed of a beta-sheet structure and α-synuclein [Johansson, J. Swiss Med. WKLY 133:275-282 (2003)].

It should be further noted that proteins having alpha-helix structure that may be converted under certain conditions to beta-sheet structure, may be also applicable by the method of the invention. Such proteins may be for example the lung surfactant associated protein C (SP-C), the amyloid beta-peptide (Aβ), proteoglycans, α₁-antichemotrypsin, apolipoprotein E and the serum amyloid P component (SAP) [Johansson (2003) ibid.].

By “sufficient amount of a protein which has a beta-sheet conformation” as used herein is meant any amount sufficient for enhancing aggregate formation that may be detected by the method of the invention.

It should be appreciated that different amounts would be needed for different beta-sheet proteins, and also for different samples obtained from different subjects. For example, the amount of a beta-sheet protein sufficient for detection of prion proteins aggregates in urine sample of a cow should be higher than the amount necessary for the detection of prion aggregates in urine samples obtained from humans.

As a non-limiting example, where the beta-sheet protein is the IgG light chain, sufficient amount may range between 10 to 0.05 microgram per sample, preferably, between 5 to 0.5, and most preferably, between 2 to 1 microgram per sample. As indicated in the examples, the amount of IgG LC used for 5 ml urine sample obtained from sheep and cows, was 1.65 microgram.

It should be appreciated that addition of a protein which has a beta-sheet conformation to the tested sample, may also enable the detection of aggregates formed in a positive sample. As shown by FIGS. 1, 3 and 4, IgG LC, recombinant BJ protein and the recombinant mouse PrP protein, which were added to the samples as a beta-sheet proteins, became protease resistant in samples obtained from subjects having a prion disease, probably due to the aggregate formation. Therefore, detection of prion diseases related aggregates, by the detection of the beta-sheet protein which was added to the sample for enhancing aggregate formation, is feasible. However, as shown by FIGS. 1 to 4, in order to distinguish between a normal sample and a sample obtained from a subject which has a prion disease, proteinase K digestion is needed. These results indicate that clear detection of the prion disease related aggregates by measuring the added beta-sheet proteins, require separation of these aggregates from the mixture. Such separation may be achieved by proteinase K digestion or by any other suitable means.

Therefore, the present invention further provides a method for the diagnosis of a neurodegenerative disorder in a mammalian subject comprising: (a) providing a body fluid sample of said subject; (b) concentrating proteins comprised within the sample, by a suitable means; (c) contacting the concentrated sample obtained in step (b) with a sufficient amount of a protein which has a beta-sheet structure, performed under conditions suitable to allow the formation of aggregates, comprising a protein associated with a neurodegenerative disorder; (d) separating said aggregates formed in step (c) from the mixture by a suitable means and (e) measuring aggregate formation by suitable means, whereby the presence of aggregates in the sample indicates that the tested subject carries said neurodegenerative disorder. More specifically, separation of aggregates from the mixture may be performed by proteinase K digestion as shown by Example 1. Alternatively, aggregates may be separated from the mixture by filtration, for example using Amicon filters (e.g., filters having cutoff of about 30 Kd), which are most preferred, or alternatively, by dialysis and centrifugation. The separated aggregates may be then measured by adding a binding material capable of binding aggregates of proteins associated with a neurodegenerative disorder, which binding material may be an antibody, a peptide, a substance having affinity to a specific compound in said aggregate or a specific dye (Congo Red, for example). The samples are subsequently applied onto a solid support. The detection of a visual signal indicates the presence of aggregates comprising a neurodegenerative disorder associated protein in the tested sample. As shown by Example 1, detection of the separated aggregates may be performed using an antibody which specifically recognizes the beta-sheet protein which was added to the sample for enhancing aggregate formation. For example, an anti human IgG antibody, or anti PrP antibody, 6H4 (as shown by FIG. 4).

Alternatively, the beta-sheet protein added may be labeled for example, by biotin as shown by FIG. 3, and then, the aggregates may by detected using avdin. It is to be appreciated that in addition to the biotin-avidin system other high affinity systems may be used for detection of the beta-sheet protein. Such systems include as non-limiting example the GST-glutathione system and CBD-cellulose.

According to another preferred embodiment, the method of the invention is intended for diagnosis of a neurodegenerative disorder, preferably disorder related to amyloidosis or a conformational disease. The term “conformational diseases” refers to that group of disorders arising from propagation of an aberrant conformational transition of an underlying protein, leading to protein aggregation and tissue deposition. Such diseases can also be transmitted by an induced conformational change, propagated from a pathogenic confomer to its normal or non-pathogenic conformer and in this case they are called herein “transmissible conformational disease”. Examples of such diseases are Alzheimer's disease, multiple sclerosis, or spongiform encephalopathy. More specifically, spongiform encephalopathy may be any one of Creutzfeldt-Jakob disease (CJD), Gerstmann-Straussler-Scheinker Syndrome (GSS), Kuru and FFI (Fatal Familial Insomnia) in humans, scrapie in sheep and goats and bovine spongiform encephalopathy (BSE) in cattle, spongiform encephalopathy of exotic ruminants (nyala, gemsbok, Arabian oryx, eland, kudu, scimitar-homed oryx, ankole, and bison); feline spongiform encephalopathy (domestic cat, puma, cheetah, ocelot, tiger), CWD (Chronic Wasting Disease) of mule, deer and elk and TME (Transmissible Mink Encephalopathy).

The term “Gerstmann-Strassler-Scheinker Disease” abbreviated as “GSS” refers to a form of inherited human prion disease. The disease occurs from an autosomal dominant disorder. Family members who inherit the mutant gene succumb to GSS.

Still further, the method of the invention is particularly applicable for mammalian subjects such as humans, sheep, goats, bovines, minks, hamsters and felines such as cats.

The body fluid sample used by the method of the invention may be a sample of blood, lymph, milk, urine, faeces, ocular fluids, saliva, semen, brain extracts, spinal cord fluid (SCF), appendix, spleen and tonsillar tissue extracts. Preferred sample may be a urine sample.

It should be appreciated that although a preferred sample may be a body fluid sample, the method of the invention may be applicable for any sample.

Therefore, the term “sample” refers to any cell, tissue, or fluid from a biological source, or any other medium that can advantageously be evaluated in accordance with the invention including, but not limited to, a biological sample drawn from a human patient, a sample drawn from an animal, a sample drawn from food designed for human consumption, a sample including food designed for animal consumption such as livestock feed, an organ donation sample, or the like.

It should be noted that according to a specific embodiment, the proteins in the tested sample are concentrated by centrifugation and precipitation.

Preferably, as indicated in the following examples, the sample is collected and centrifuged, the resulting supernatant is then collected, a suitable buffer is added to said supernatant for a suitable time period, followed by centrifugation, and supernatant collection. For precipitation, MeOH is added to the sample. It is to be appreciated that any other suitable protein precipitation methods such as TCA (Trichloracetic acid), may be used by the method of the invention.

According to another specific embodiment, wherein the neurodegenerative disorder diagnosed by the method of the invention is a prion disease, the neurodegenerative disorder associated protein may be the abnormal isoform of prion protein (PrP^{SC}).

PrP^{SC} is the major constituent of the pathogenic amyloid plaques that are found in the brains of many hosts with spongiform encephalopathies. The quantity of this protein correlates with the titer of prion infectivity in brain. Moreover, PrP^{SC} was absent from uninfected brain, and it was found that various procedures that denatured, hydrolysed, or modified PrP also inactivated prion infectivity.

No differences in the primary structure (i.e. amino acid sequence) of PrP^{C} and PrP^{SC} have been detected, nor have any differences been found between PrP genes or mRNAs from normal and infected brains with respect to structure or copy number. The physical differences such as three-dimensional configuration between the two proteins are therefore attributed to post-translational chemical modification. In general, during the refolding of PrP^{C} into PrP^{SC}, some of the normal α-helical protein structure is partially converted into beta-sheet.

The present invention thus provides for a method for the diagnosis of spongiform encephalopathy, such as Creutzfeldt-Jakob disease (CJD), Gerstmann-Straussler-Scheinker Syndrome (GSS), Kuru, scrapie or bovine spongiform encephalopathy (BSE), in a mammalian subject. According to a particular embodiment, this method comprises: (a) providing a urine sample of said subject; (b) concentrating proteins comprised within the sample by using Amicon tubes or by centrifugation and precipitation; (c) contacting the concentrated sample obtained in step (b) with a sufficient amount of IgG LC, under conditions suitable to allow the formation of aggregates comprising the abnormal isoform of prion protein (PrP^{SC}); (d) adding Congo Red to the sample mixture obtained in step (c), in an amount sufficient for detection of aggregates comprising the abnormal isoform of the prion protein (PrP^{SC}); (e) applying the sample obtained in step (d) onto a nitrocellulose membrane; and (f) detecting a visual signal indicating the presence of aggregates comprising the abnormal isoform of prion protein (PrP^{SC}) in the tested urine sample; whereby the presence of aggregates in the sample indicates that the tested subject may carry a prion disease.

It should be appreciated that diagnosis of prion disease according to the method of the invention may be used for diagnosing a prion disease in a human or animal subject, by obtaining a urine sample of the subject and detecting the presence of the abnormal isoform of prion protein (PrP^{SC}) in said urine sample by the detection method the invention, the presence of the PrP^{SC} protein in the urine of the subject indicating that said subject carries a prion disease. This abnormal isoform is probably a pathogenic isoform of the prion protein. Thus, the invention provides a method for the detection of different prion diseases before or after onset of clinical symptoms.

The diagnostic method of the invention is particularly important for detecting carriers of CJD, for monitoring treatment of CJD patients and for estimating the clinical stage as well as the severity of the disease. It is to be noted that when referring to CJD, all other TSE's are also included. Suspected carriers of pathogenic prion mutations are tested by molecular method for the presence of the mutation, which defines their carrier status. However, and since the age of disease onset can be between 35-85 years or more, there is no test to establish at early stages whether the disease is manifesting. Such test could be crucial for early or prophylactic treatment. The detection of carriers of the mutation leading to CJD disease may be used, for example, in genetic counseling.

Additionally, the diagnostic method of the invention is useful in identifying infection of BSE, particularly in individuals that have been exposed to the disease. Identifying human carriers of BSE has importance, inter alia, in screening blood samples of human donors for the presence of a prion disease in the donors. Screening can be carried out, for example, by obtaining a urine sample from the donor, detecting the presence of the abnormal isoform of prion protein (PrP^{SC}) in the urine sample by the detection method of the invention and ascribing the results of the detection to said blood sample. Such screening would prevent the use of prion-infected blood, thus diminishing risks of blood transfusions.

Additionally, the diagnostic method of the invention, when applied to bovine animals, and also to other domestic animals like sheep and goats or any other animal of interest susceptible to BSE or any other prion disease, may assist in screening food products originating from the tested animals, like meat and dairy products, and reduce the risk of infection of human consumers.

In a further aspect, the present invention relates to a method for detecting the presence of a neurodegenerative disorder associated protein in a sample of a subject, such method comprises the steps of: (a) providing a body fluid sample of the tested subject; (b) concentrating proteins comprised within the sample by suitable means, preferably by centrifugation and precipitation; (c) contacting the concentrated sample obtained in step (b) with a sufficient amount of a protein which has a beta-sheet structure, under conditions suitable to allow the formation of aggregates comprising the neurodegenerative disorder associated protein; (d) measuring aggregate formation by suitable means.

According to a particular embodiment, the measurement of aggregate formation in step (d) comprises the following steps: (i) adding to the mixture obtained in step (c), a binding material capable of binding aggregates of proteins associated with neurodegenerative; (ii) applying the sample obtained in step (i) onto a solid support; and (iii) detecting a visual signal indicating the presence of aggregates comprising the neurodegenerative disorder associated protein in the tested sample.

According to another particular embodiment, the method of the invention may optionally further comprise the step of separating said aggregates from said mixture by a suitable means, prior to addition of said binding material. Such suitable means may be for example, proteinase K digestion or alternatively, dialysis and centrifugation.

According to a specific embodiment, the binding material may be an antibody, a peptide, a substance having affinity to a specific compound in said aggregate or a specific dye. Preferably, a specific dye that may be any one of Congo Red, Thioflavin-T and Thioflavin-S. Most preferably, the binding material may be Congo Red. Alternatively, the binding material may be an antibody which specifically recognizes said protein which has a beta-sheet structure.

In another specific embodiment, the method of the invention utilizes any one of IgG light chain (LC), human Bence Jones (BJ) protein and recombinant PrP protein as a protein which has a beta-sheet structure. Preferably, IgG light chain (LC) is used.

According to a particular and preferred embodiment, the invention specifically provides a method for detecting the presence of the abnormal isoform of prion protein (PrP^{SC}) in a urine sample of a subject. This method comprises the steps of: (a) providing a urine sample of the tested subject; (b) concentrating proteins comprised within the sample by amicon tubes or by centrifugation and precipitation; (c) contacting the concentrated sample obtained in step (b) with a sufficient amount of IgG LC, under conditions suitable to allow the formation of aggregates comprising the abnormal isoform of prion protein (PrP^{SC}); (d) adding Congo Red to the sample mixture obtained in step (c), in an amount sufficient for detection of formation of aggregates comprising the abnormal isoform of prion protein (PrP^{SC}); (e) applying the sample obtained in step (d) onto a nitrocellulose membrane; and (f) detecting a visual signal indicating the presence of aggregates comprising the abnormal isoform of prion protein (PrP^{SC}) in the tested urine sample; whereby the presence of aggregates in the analyzed sample is indicative of the presence of the abnormal isoform of prion protein (PrP^{SC}) in said sample.

In a third aspect, the present invention relates to kit for the diagnosis of a neurodegenerative disorder in a mammalian subject, such kit comprising: (a) means for obtaining a sample from a tested mammalian subject; (b) means for concentrating proteins in the tested sample; (c) composition containing a protein which has a beta sheet structure, for enhancing aggregate formation; (d) means for measuring aggregate formation in the sample; (e) optionally, suitable buffers; and (f) instructions for carrying out the detection of the presence of aggregates comprising a neurodegenerative disorder associated protein in the tested sample.

According to one embodiment, the kit of the invention may optionally further comprise means for separating the aggregates from the sample prior to measuring aggregate formation.

According to another embodiment, the kit of the invention may comprise as means for measuring aggregate formation, a binding material capable of binding a neurodegenerative disorders related aggregate.

More particularly, such binding material may be a specific dye such as Congo Red, Thioflavin-T or Thioflavin-S. Preferably, such specific dye may be Congo Red.

Alternatively, such binding material may be an antibody which specifically recognizes the protein which has a beta-sheet structure added to the sample.

Where the detection of aggregates in a sample performed uses a specific dye such as Congo Red or an antibody, the kit of the invention may also comprise solid support for attachment of proteins in said sample. Such support may be, for example, nitrocellulose membrane. As indicated by the Examples, the samples are applied onto nitrocellulose and a dot blot assay is performed. It should be noted that any other suitable solid support may be applicable.

In another specifically preferred embodiment, the kit of the invention comprises a composition containing any one of IgG light chain (LC), human Bence Jones (BJ) protein and recombinant PrP protein, preferably, IgG light chain (LC), as a protein which has a beta-sheet structure.

The kit of the invention is intended for the diagnosis of a neurodegenerative disorder such as Alzheimer's disease, multiple sclerosis, and spongiform encephalopathy, and is useful in carrying out all of the diagnostic methods of the invention.

The invention further provides for the use of a protein which has a beta-sheet structure, which enhances the formation of aggregates comprising a neurodegenerative disorder associated protein, in the preparation of a diagnostic composition for the diagnosis of a neurodegenerative disorder.

In one embodiment, the protein which has a beta-sheet structure may be IgG light chain.

According to another preferred embodiment, where the neurodegenerative disorder to be diagnosed is spongiform encephalopathy, the disease associated protein may be the abnormal isoform of prion protein (PrP^{SC}).

Therefore, the invention provides for a diagnostic composition for the detection of a neurodegenerative disorder in a mammalian subject, preferably, spongiform encephalopathy. Such composition comprises as an effective ingredient a sufficient amount of a protein which has a beta-sheet structure.

According to a preferred embodiment, the composition of the invention may comprise any one of IgG light chain (LC), human Bence Jones (BJ) protein and recombinant PrP protein, preferably, IgG LC as an active ingredient.

The invention further provides for a method for the preparation of a diagnostic composition for the detection of a neurodegenerative composition in a mammalian subject. Such method comprises the step of (a) providing a protein which has a beta-sheet structure and enhances the formation of aggregates comprising a neurodegenerative disorder associated protein; and (b) adding a binding material capable of binding a neurodegenerative diseases related aggregate to a sample containing the protein of (a).

A number of methods of the art of molecular biology are not detailed herein, as they are well known to the person of skill in the art. Such methods include, for example, detection and analysis of naturally occurring, synthetic and recombinant proteins or peptides and the like. Textbooks describing such methods are e.g., Sambrook et al., Molecular Cloning A Laboratory Manual, Cold Spring Harbor Laboratory; ISBN: 0879693096, 1989, Current Protocols in Molecular Biology, by F. M. Ausubel, ISBN: 047150338X, John Wiley & Sons, Inc. 1988, and Short Protocols in Molecular Biology, by F. M. Ausubel et al. (eds.) 3rd ed. John Wiley & Sons; ISBN: 0471137812, 1995. These as well as all other publications cited herein, are incorporated herein in their entirety by reference, including references cited therein. Furthermore, a number of immunological techniques are not in each instance described herein in detail, as they are well known to the person of skill in the art. See e.g., Current Protocols in Immunology, Coligan et al. (eds.), John Wiley & Sons. Inc., New York, N.Y.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the intended scope of the invention.

### EXAMPLES

Experimental Procedures

Proteins Having a Beta-Sheet Conformation

Human Light chain Lambda (3.3 mg/ml) obtained from Sigma [CAT NO. I 5029].

Bence Jones protein—λ light chain of human BJ protein 10 μg was purchased from SIGMA Israel Cat No. IAB74179. The BJ protein was biotinylated by SIGMA.

Mouse recombinant PrP—construct of the full length PrP was a kind gift from Dr. Gasset Maria [Instituto Quimica-Fisica ‘Rocasolano’, CSIC, Serrano 119, E-28006, Madrid, Spain].

Antibodies

6H4—anti PrP^{Sc}monoclonal antibody, which binds to the amino acid sequence of residues 144-152 of the PrP protein, purchased from Prionics AG, Switzerland.

AP conjugated anti-human IgG—was purchased from Jackson research Cat. No. 109-055-003

Biotinylation

was performed using the NHS—Biotin kit—purchased from Pierce Cat. No. #20217 according to the manufacturer instructions.

Buffers

STE:

Tris HCl pH7.5, 10 mM

NaCl 10 mM

EDTA 1 mM pH 8

Membranes for Dot Blot

Nitrocellulose membrane: Schleicher & Schuell Protran BA83, Cellulose nitrate 0.2 μl.

Amicon tubes cat. No. 9031 MINICON B15 were used for concentration of 5 ml urine samples.

Protocol for Prion Detection Method of the Invention: Improved Congo Red Staining Dot-Blot

5 ml urine samples obtained from different tested subjects were centrifuged for 1 min. at 4000 rpm, and the supernatant was kept.

2 ml of 0.5 M EDTA (pH 8) were added to the supernatant and the sample was rotated for 1 hour at room temperature, followed by centrifugation for 1 hour at 4000 rpm. The supernatant was kept, and 30 ml of MeOH were added to the supernatant for 1 hour at −70° C. or overnight at −20° C. Samples were then centrifuged for 30 min. at 4000 rpm and the pellet was dried and kept. The pellet was then re-suspended with 50 μl of STE+2% sarcosyl and 0.5 μl of human Light Chain Lambda was added. Samples were incubated for 1 hour at room temperature or overnight at 4° C., followed by centrifugation for 2 min. at 4000 rpm to get a clear sample.

10 μl of the sarcosyl solution were added to 10 μl of sample, mixed by pipetting, and then 2 μl of Congo Red fresh solution were added to a final concentration of 200 μg/ml (stock solution: 2 mg/ml DDW) and incubated for 1 hour or more. 2 μl of the mixture were applied to a nitrocellulose membrane and the membrane was dried and subjected to washes as follows:

1 min. with DDW;

1 min. with 50% MeOH in DDW;

1 min. with 70% MeOH;

1 min with 80% MeOH;

Incubation in 90% MeOH until the negative control disappears.

It may be required to increase the methanol concentration up to 94%.

Western Blot Analysis

Samples boiled in SDS sample buffer were applied to 12% or 14% SDS PAGE and subsequently transferred to a nitrocellulose membrane. Membranes were blocked with 3% fat milk except for the bovine samples which were blocked with 5% HSA (Human Serum Albumin, Sigma). Membranes were then rinsed in TBST for 15 min and immunoblotted using the specific antibodies as indicated.

### Example 1

IgG Light Chain is Present and Becomes Protease Resistant in Urine Samples of Infected Subjects

In search for improved, sensitive and specific non-invasive diagnostic methods for the detection of the prion protein PrP in urine samples of different subjects, the inventors developed a protocol for TSE urine testing, based on Congo Red (CR) staining of urine prion aggregates. In this test, urine from CJD patients and scrapie infected hamsters could be stained with CR following concentration of the urine sample, and identified as positive by a dot blot protocol. However, this method was not applicable to bovine and sheep samples.

Immunoblot experiments performed by the inventors using urine samples obtained from humans and hamsters affected with prion diseases have shown that in some samples, the secondary anti-mouse antibody by itself also reacts with the sample in a disease-specific manner.

In order to further examine this phenomena, the inventors compared protein profile of urine samples obtained from normal and scrapie infected sheep. As shown by the table presented in FIG. 1, which schematically illustrates the protein profile of the examined samples, urine samples obtained from normal or scrapie infected sheep, were concentrated as described in Experimental Procedures, and were separated on 12% SDS PAGE, before and after proteinase K (PK) digestion. The gels were subsequently stained with Coomassie Blue, and bands were cut from the gel and sequenced by mass spectrometry. The results of the protein sequencing, which are illustrated in the table showed by FIG. 1, clearly indicate that the dominant protein present specifically in scrapie sheep samples, is the IgGκ chain α-1. It should be noted that this protein was resistant to PK.

The existence of PK resistant IgG light chain (LC) in samples obtained from prion infected subjects was further examined in CJD patients. Urine samples from patients and controls were concentrated by minicon and digested in the presence or absence of PK. All samples were subjected to SDS-PAGE and immunoblotted with anti-human IgG conjugated with AP (alkaline phosphatase). As shown by FIG. 2, PK resistant IgG light chain was detected in most of the urine samples obtained from patients (only the patient sample #17 was negative) and also in both healthy carriers of the E200K mutation. These results suggest that IgG light chain in urine of prion diseased individuals is resistant to protease cleavage. Moreover, these results clearly indicate the feasibility of using LC IgG, as a reliable marker for prion diseases.

In order to examine the possibility that IgG light chain becomes protease resistant in urine of prion diseased subjects, the inventors next examined the ability of urine samples of CJD patients to render Bence Jones (BJ) protein, which is considered to be amyloidogenic LC, protease resistant. Therefore, urine samples from Multyple Myeloma patient was concentrated by minicon and digested in the presence or absence of 20 μg/ml PK for 30 min. at 37° C. Samples were subjected to SDS PAGE and either stained with CB or immunoblotted with anti human IgG. As shown by FIG. 3A, Bence Jones (BJ) protein, which is the main protein present in multyple myeloma samples is not proteases resistant even when present at very large concentrations. The inventors next examined the ability of CJD urine samples to incorporate beta sheet proteins, such as the BJ protein into an existing seed of aggregation, and thereby render such proteins PK resistant. Therefore, biotinylated purified BJ proteins were incubated overnight alone or in the presence of 5 ml CJD or AD urine as a negative control. Samples were concentrated by minicon, digested in the presence and absence of PK (20 μg/ml for 30 min at 37° C.), and subsequently immunoblotted either with anti human IgG or with Avidin AP.

As shown by FIG. 3B, only the BJ proteins incubated with CJD urine became protease resistant.

The inventors next examined the possibility that other proteins having a beta-sheet structure may become protease resistant in prion diseased urine samples. As shown by FIG. 4, mouse recombinant PrP was incubated overnight with concentrated normal or CJD urine and subsequently, the samples were digested in the presence of PK. The results clearly indicate that recombinant PrP became protease resistant in the presence of prion urine.

These results may suggest that light chain IgG (LC), present in the urine samples of affected subjects, can incorporate into a specific prion aggregate which comprises the PrP protein. Without being bound by theory, this phenomenon can be explained by the fact that similarly to the prion protein PrP, LC is known to fold into a beta-sheet conformation. Therefore, addition of protein which has a beta-sheet conformation to a sample containing the prion protein may enhance aggregation and thus increase the signal.

### Example 2

IgG Light Chain Increases Prion Aggregates in Urine Samples of Infected Subjects

Based on the findings detailed above, the inventors further developed a new protocol for a sensitive and specific detection of PrP protein in prion infected samples. This protocol is based on the addition of external protein which has a beta-sheet structure, preferably, IgG light chain (LC), to the concentrated urine samples. Following incubation for 2 to 20 h, Congo Red (CR) is added and the samples are subjected to a dot blot assay as indicated in the experimental procedures.

As shown by FIG. 5, urine samples obtained from infected bovine (BSE) where stained with CR only in the presence of human IgG LC. No signal was seen when normal control samples were used. Furthermore, LC addition resulted in an enhancement of the signal in samples obtained from CJD human patients. Without being bound by the theory, these results may possibly indicate that there is an optimal LC concentration required for the CR staining. The LC concentration in bovine and sheep urine samples is lower and therefore, addition of external LC to the sample induces aggregation and thus enables detection of the enhanced signal by the method of the invention.

### Example 3

Early Diagnosis of CJD by the Detection Method of the Invention

As shown above, the method of the invention enables sensitive detection of prion-infected subjects such as bovine. Therefore, the inventors next examined whether addition of beta-sheet protein (e.g., LC) further provides enhanced signal in CJD samples. Familial CJD is a dominant disorder and therefore the defected gene is usually transmitted to 50% of the offsprings. The disease appears late in life, from the age of 40 onwards. In order to establish whether this improved test may be used for early diagnosis, the inventors tested whether CR may stain the urine of CJD carriers. As shown by FIG. 6, CJD patients (samples 1, 12, 19 and 23) and family members of one of the patients were tested by the CR method of the invention. As expected, samples obtained from patients, and suspected patients were positive, most of the mutation carriers were detected, and none of the control samples (healthy individuals) were stained. It should be noted that only part of the healthy carriers were positive, suggesting that the test of the invention indicates (contrary to the genetic test) that only part of the carriers are close to getting the disease while the negative carriers are still out of risk. It should be further noted that one out of eight samples obtained from non-carriers (sample 3) was also positive. This may be due to interfamily contamination or to false positive results in human urine, which may be caused in cases of urinary track infection or severe kidney dysfunction.

### Example 4

Detection of Prion Protein in Scrapie and Bovine Samples by the Method of the Invention

In order to examine the specificity and sensitivity of the detection method of the invention, double blind test was performed in 32 samples obtained from scrapie infected and non-infected sheep received from the VLA [Department of Agriculture] in England (FIG. 7) and 16 bovine samples (FIG. 8).

As shown by FIG. 7, all known scrapie samples, except sample 12, were identified by the test and four out of eight suspected samples were identified and developed prion disease one month later. All 14 negative samples (obtained from sheep of infected or clean flocks), were found to be negative by the test of the invention.

FIG. 8 shows that by using the diagnostic method of the invention, all samples obtained from BSA infected cows, were identified correctly.

1-57. (canceled)

58. A method for the diagnosis of a neurodegenerative disorder in a mammalian subject comprising:
a. providing a body fluid sample of said subject;
b. concentrating proteins comprised within said sample by suitable means;
c. contacting the concentrated sample obtained in step (b) with a sufficient amount of a protein which has a beta-sheet structure, under conditions suitable to allow the formation of aggregates, said aggregates comprise a protein associated with said neurodegenerative disorder; and
d. measuring aggregate formation by suitable means, whereby the presence of aggregates in said sample indicates that said subject carries said neurodegenerative disorder.

59. The method according to claim 58, wherein the measurement of aggregate formation in said step (d) comprises the following steps:
(i) adding to the mixture obtained in step (c) a binding material capable of binding aggregates of proteins associated with said neurodegenerative disorder;
(ii) applying the sample obtained in step (i) onto a solid support; and
(iii) detecting a visual signal which indicates the presence of aggregates comprising a neurodegenerative disorder-associated protein in said tested sample.

60. The method according to claim 59, further comprising the step of separating said aggregates from said mixture by suitable means, prior to the addition of said binding material, wherein said suitable means is selected from the group consisting of proteinase K digestion, dialysis and centrifugation.

61. The method according to claim 59, wherein said binding material is selected from the group consisting of an antibody, a peptide, a substance having affinity to a specific compound in said aggregate and specific dye.

62. The method according to claim 61, wherein said binding material is an antibody which specifically recognizes said protein which has a beta-sheet structure.

63. The method according to claim 58, wherein said protein which has a beta-sheet structure is selected from the group consisting of IgG light chain (LC), human Bence Jones (BJ) protein and recombinant PrP protein, preferably, IgG light chain (LC).

64. The method according to claim 58, wherein said neurodegenerative disorder is any one of Alzheimer's disease, multiple sclerosis, and spongiform encephalopathy selected from Creutzfeldt-Jakob disease (CJD), Gerstmann-Straussler-Scheinker Syndrome (GSS), Kuru, scrapie and bovine spongiform encephalopathy (BSE).

65. The method according to claim 64, wherein said mammalian subject is selected from the group consisting of humans, sheep, goats, bovines, minks, hamsters and cats.

66. The method according to claim 58, wherein said body fluid sample is selected from the group consisting of: blood, lymph, milk, urine, faeces, semen, brain extracts, spinal cord fluid (SCF), appendix, spleen and tonsillar tissue extracts samples.

67. The method according to claim 58, wherein concentrating the proteins in said sample is performed by centrifugation and precipitation.

68. The method according to claim 58, wherein said neurodegenerative disorder-associated protein is the abnormal isoform of prion protein (PrP^{SC}).

69. A method according to claim 58, for the diagnosis of a spongiform encephalopathy in a mammalian subject comprising:
(a) providing a urine sample of said subject;
(b) concentrating proteins comprised within said sample;
(c) contacting the concentrated sample obtained in step (b) with a sufficient amount of IgG LC, under conditions suitable to allow the formation of aggregates, which aggregates comprise the abnormal isoform of prion protein (PrP^{SC});
(d) adding Congo Red to the sample mixture obtained in step (c), in an amount sufficient for detection of aggregates comprising the abnormal isoform of the prion protein (PrP^{SC});
(e) applying the sample obtained in step (d) onto a nitrocellulose membrane; and
(f) detecting a visual signal indicating the presence of aggregates comprising the abnormal isoform of prion protein (PrP^{SC}) in said tested urine sample; whereby the presence of said aggregates in said sample indicates that said subject carries a prion disease.

70. The method according to claim 69, wherein diagnosis of said spongiform encephalopathy is performed prior to or after onset of clinical symptoms.

71. A method for detecting the presence of a neurodegenerative disorder-associated protein in a sample of a subject, said method comprising the steps of:
(a) providing a body fluid sample of said subject;
(b) concentrating proteins comprised within said sample by a suitable means;
(c) contacting the concentrated sample obtained in step (b) with a sufficient amount of a protein which has a beta-sheet structure, under conditions suitable to allow the formation of aggregates, which aggregates comprising said neurodegenerative disorder-associated protein; and
(d) measuring aggregate formation by suitable means.

72. The method according to claim 71, for detecting the presence of the abnormal isoform of prion protein (PrP^{SC}) in a urine sample of a subject, said method comprising the steps of:
(a) providing a urine sample of said subject;
(b) concentrating proteins comprised within said sample;
(c) contacting the concentrated sample obtained in step (b) with a sufficient amount of IgG LC, under suitable conditions allowing the formation of aggregates comprising the abnormal isoform of prion protein (PrP^{SC});
(d) adding Congo Red to the sample mixture obtained in step (c), in an amount sufficient for detection of formation of aggregates which comprise the abnormal isoform of prion protein (PrP^{SC});
(e) applying the sample obtained in step (d) onto a nitrocellulose membrane; and
(f) detecting a visual signal indicating the presence of aggregates comprising the abnormal isoform of prion protein (PrP^{SC}) in said tested urine sample; whereby the presence of said aggregates in said sample is indicative of the presence of the abnormal isoform of prion protein (PrP^{SC}) in said sample.

73. A kit for the diagnosis of a neurodegenerative disorder in a mammalian subject, comprising:
(a) means for obtaining a sample from a tested mammalian subject;
(b) means for concentrating proteins in said sample;
(c) a protein which has a beta sheet structure;
(d) means for measuring aggregate formation in said sample;
(e) optionally, suitable buffers;
(f) instructions for carrying out the detection of the presence of aggregates comprising a neurodegenerative disorder-associated protein in said sample; and
(g) optionally, means for separating said aggregates from said sample prior to measuring aggregate formation.

74. The kit according to claim 73, wherein said means for measuring aggregate formation is a binding material capable of binding said neurodegenerative disease associated protein aggregate, preferably, said binding material is selected from the group consisting of an antibody, a peptide, a substance having affinity to a specific compound in said aggregate and a specific dye.

75. The kit according to claim 72, wherein said binding material is any one of Congo Red, Thioflavin-T and Thioflavin-S.

76. The kit according to claim 72, wherein said binding material is an antibody which specifically recognizes said protein which has a beta-sheet structure.

77. The kit according to claim 71, further comprising solid support for binding proteins in said sample.

78. The kit according to claim 71, wherein said protein which has a beta-sheet structure is selected from the group consisting of IgG light chain (LC), human Bence Jones (BJ) protein and recombinant PrP protein.

79. The kit according to claim 78, wherein said neurodegenerative disorder is any one of Alzheimer's disease, multiple sclerosis, and spongiform encephalopathy selected from, Creutzfeldt-Jakob disease (CJD), Gerstmann-Straussler-Scheinker Syndrome (GSS), Kuru, scrapie and bovine spongiform encephalopathy (BSE).

80. The kit according to claim 79, wherein said mammalian subject is selected from the group consisting of humans, sheep, goats, bovines, minks, hamsters and cats.

81. The kit according to claim 80, wherein said body fluid sample is selected from the group consisting of blood, lymph, milk, urine, faeces, semen, brain extracts, spinal cord fluid (SCF), appendix, spleen and tonsillar tissue extracts samples.

82. The kit according to claim 81, wherein said neurodegenerative disorder-associated protein is the abnormal isoform of prion protein (PrP^{SC}).

83. A diagnostic composition for the detection of a neurodegenerative disorder in a mammalian subject, which composition comprises as an effective ingredient a sufficient amount of a protein which has a beta-sheet structure.
